(19) United States
(12) Patent Application Publication
(10) Pub. No.: US 2009/0326420 A1
(43) Pub. Date: Dec. 31, 2009

(54) DEVICE FOR THE VOLUME TREATMENT OF BIOLOGICAL TISSUE

(76) Inventors: Chrétien Moonen, Gradignan (FR); Charles Mougenot, Bordeaux (FR)

Correspondence Address: BLAKELY SOKOLOFF TAYLOR & ZAFMAN LLP, 1279 OAKMEAD PARKWAY, SUNNYVALE, CA, 94085-4040, US

(21) Appl. No.: 12/438,934

(22) PCT Filed: Aug. 13, 2007
(86) PCT No.: PCT/EP07/58361

§ 371 Date: Feb. 25, 2009

(30) Foreign Application Priority Data
Aug. 29, 2006 (FR) 0607584

(51) Int. Cl. A61N 7/02

(52) U.S. Cl. 601/3

(57) ABSTRACT

The invention relates to a device for the heat treatment of a target region of a biological tissue, comprising: energy generator means for delivering energy to a focal point (P) in the target region; means for measuring the spatial temperature distribution in said region; a control unit comprising means for controlling the displacement of the focal point (P) towards successive treatment points and means for controlling the energy to be delivered by the generator means to the successive treatment points, characterized in that the control unit further includes means for determining a spatial and energy distribution of the treatment points where treatment is to be carried out, each successive treatment point having a position and a treatment energy that are determined according to the positions and spatial and energy distributions of the previous treatment points. The invention also relates to the associated heat treatment method.

### AREA OF THE INVENTION

The invention concerns the area relating to the treatment of biological tissue using hyperthermia.

### STATE OF THE ART

Therapies using local hyperthermia consist in locally heating a target area or region of a biological tissue. When this type of therapy is used for gene therapy, the heat may be used for its action on a heat-sensitive promoter for example. Heat can also be used to necrotize biological tissue and for tumour ablation.

Therefore, therapies using local hyperthermia offer numerous advantages. These advantages are both qualitative and economy-related. From a qualitative viewpoint, they offer strong potential for controlling treatments such as gene therapy, the local depositing of medication, tumour ablation. From an economic viewpoint, they are compatible with ambulatory treatment of patients, allowing the length of hospital stays to be reduced, etc.

For therapies using hyperthermia, heat may be for instance provided by a laser, microwaves or radiofrequency waves, focused ultrasound, etc. Generally speaking, therapies using local hyperthermia allow surgical acts to be performed whose invasiveness is reduced to a minimum. However, amongst the types of above-mentioned energy sources, focused ultrasound is of particular interest since it allows the focused area to be heated non-invasively, deep inside a biological body, without significantly heating tissues adjacent to the focused area.

In all cases, the temperature of the area and its immediate vicinity, during treatment, must be precisely and continuously controlled. For this purpose, Magnetic Resonance Imaging can be used (MRI). With MRI it is possible to achieve accurate mapping of temperature distributions and detailed anatomical information. In addition, by controlling the heating tool, non-invasive temperature control can be obtained which, with ultrasound, makes the treatment device fully non-invasive.

Devices are already known for point temperature control during treatment with focused ultrasound, which are based on magnetic resonance thermometry.

The size of the tumours to be treated is generally much larger than the size of the focal point. At a frequency of 1.5 Mhz, the size of the focal point is close to the wavelength i.e. 1 mm in biological organs. On the other hand, cancerous tumours which can be detected by MRI are closer to one centimetre in size. Although chemotherapy allows the size of these tumours to be reduced, treatment using point ultrasound is insufficient.

The technique of point temperature control can be used successively over several points. However, this protocol is very time-consuming, since a wait of several minutes is required between each point so that the temperature returns to its initial state. Also, there is a risk that tumour cells located between two target points may not be necrotized.

It has been subsequently envisaged to carry out spatial control over temperature, using a technique similar to point temperature control, with the exception that several points are heated simultaneously. One solution for example was put forward by French patent application n^{o} 04-04562 filed on 24 Apr. 2004 titled: “Heat treatment assembly for biological tissues”, which provided temperature control according to a specific plan of the target region. Said solution nevertheless entails major directional constraints for focusing, and further requires long treatment times making the device little efficient.

Another system proposed in PCT application WO 02/43804 proposes monitoring the distribution of the thermal dose associated with several treatment points, with a view to choosing the positions of subsequent treatment points so as to treat areas remaining to be necrotized, the thermal dose parameters generally being constant. While said system avoids treating areas which have already attained a sufficient thermal dose for necrosis, it has a certain number of disadvantages. Firstly it is very imprecise, since the monitoring of thermal dose distribution is relatively binary i.e. it only allows determination of whether or not an area is necrotized, in order to determine whether or not the area must be further treated. Also, monitoring of thermal dose distribution implies major operating constraints which tend to slow the treatment process, since it is necessary in particular to allow complete cooling of the treated region before obtaining an accurate image of the thermal dose reached

One purpose of the present invention is to provide a device for the thermal treatment of a target region, and an associated method, allowing at least one of the above-mentioned disadvantages to be solved.

### DESCRIPTION OF THE INVENTION

For this purpose, a thermal treatment device is proposed to treat a target region of a biological tissue, comprising:
energy generating means to provide energy to a focal point P in the target region,
measuring means to measure the spatial distribution of temperature in said region,
a control unit comprising means to control movement of the focal point P towards successive treatment points, and means to control the energy to be provided by the generating means to the successive treatment points,
   characterized in that the control unit further comprises means to determine a spatial and energy distribution of the treatment points to be performed, each successive treatment point having a position and treatment energy determined in relation to the positions and spatial energy distributions of the previous treatment points.

With said device, it is therefore possible to obtain the treatment of a wide volume, which may be three-dimensional, with extensive homogeneity.

When focusing the energy source onto a spot treatment point, the ensuing spatial distribution of energy is not solely located on the treatment point but also extends to areas adjacent to the treatment point. Therefore, by taking into account previous treatment points, it is possible to adjust the position and energy intensity for the new point to be treated so as not to overexpose any area of the target region.

In this manner adverse overlapping of treatment points is avoided, which may generate overexposure.

In addition, the proposed device is highly precise since it allows the parameters for each subsequent treatment point to be set in relation to the spatial energy distribution of each previous treatment point. The spatial distribution of temperature in the region effectively allows the spatial energy distributions of the previous treatment points to be deducted, from which the spatial energy distribution remaining to be made for complete treatment of the region can be deduced. This is of particular advantage when the necrosis threshold of the target region must be reached but scarcely exceeded, as is the case with thermal dose monitoring that is relatively binary. In addition, it is not necessary to conduct any cooling whatsoever of the target region in order to monitor temperature distribution, allowing treatment time to be optimized.

Finally, by taking into account the overlap effect of spatial energy distributions, it is possible to reduce significantly the total energy used to treat a region, and hence to reduce associated risks for patients, since the treatment points located in the centre of this region are no longer necessary.

Preferred, but non-limiting, aspects of this thermal treatment device are the following:
the spatial distribution of the treatment points is three-dimensional;
the control unit comprises means to determine the spatial and energy distribution of the treatment points to be performed in relation to a spatial distribution of required energy defined by a temperature regulation system to treat the target region;
the control unit comprises means to determine the spatial distribution of required energy to treat the target region according to a Proportional-Integral-Derivative regulation system;
the means to determine the spatial and energy distribution of the treatment points to be performed comprise deconvolution means the spatial distribution of required energy to treat the target region, using a spatial energy distribution characteristic of one treatment point;
the means to determine the spatial and energy distribution of the treatment points to be performed comprise means to determine the treatment point corresponding to the maximum spatial distribution of remaining energy to treat the target region, the spatial distribution of remaining energy corresponding to the spatial distribution of required energy to treat the target region subtracted by the spatial energy distributions characteristic of the previous treatment points;
the control unit comprises means to determine the spatial and energy distribution of the treatment points to be performed, such that:

$${E}^{0} ( \vec{r} ) = {E}^{REQUIRED} ( \vec{r} ) and {Γ}^{0} ( \vec{r} ) = 0$$

$$\vert \begin{matrix}let {\vec{r}}_{n} such that {E}^{n} ( {\vec{r}}_{n} ) = max ( {E}^{n} ( \vec{r} ) ) \\ {E}^{n + 1} ( \vec{r} ) = {E}^{n} ( \vec{r} ) - R \cdot {E}^{n} ( {\vec{r}}_{n} ) \cdot {E}^{1 pt} ( \vec{r} - {\vec{r}}_{n} ) \\ {Γ}^{n + 1} ( \vec{r} ) = {Γ}^{n} ( \vec{r} ) + R \cdot {E}^{n} ( {\vec{r}}_{n} ) \cdot δ ( \vec{r} - {\vec{r}}_{n} )\end{matrix}$$

where Eⁱ(\vec{r}) corresponds to the difference between the spatial distribution of required energy and the spatial energy distributions characteristic of the previous i treatment points \vec{r}ᵢ, Γⁱ(\vec{r}) corresponds to the spatial distribution of the associated treatment points, δ is the Dirac function which is zero at every point \vec{r} different from the origin where its value is 1, R is a percentage chosen arbitrarily so that the spatial energy distribution characteristic of a treatment point is less than the spatial distribution of required energy E^{REQUIRED}(\vec{r});
the control unit comprises means to control the energy provided by the energy generating means in relation to a non-uniform temperature regimen.
the control unit comprises means to control the energy provided by the energy generating means in relation to a time-shifted temperature regimen for each treatment point.

A method is also proposed for the thermal treatment of a target region of a biological tissue, comprising the steps of:
measuring the spatial temperature distribution in said region,
commanding movement of a focal point P towards successive treatment points in the target region,
providing energy to the focal point P, characterized in that it also comprises the step of determining a spatial and energy distribution of the treatment points to be performed, each successive treatment point having a position and treatment energy determined in relation to the positions and spatial energy distributions of previous treatment points.

The proposed method of treatment can be implemented using the means of the treatment device proposed above. This treatment device further comprises means enabling implementation of other characteristic steps of the proposed treatment method, in accordance with preferred, non-limiting aspects thereof.

### DESCRIPTION OF THE FIGURES

Other characteristics and advantages will arise from the following description which is solely illustrative and non-limiting, and is to be read with reference to the appended figures amongst which:

FIG. 1 illustrates the different characteristic elements of the thermal treatment device;

FIGS. 2a to 2c illustrate the phase shifting of the electric signals to move the focal point electronically;

FIG. 3 shows the distribution of the 256 elements on the front side of a matrix transducer;

FIGS. 4a and 4b illustrate a simulation of the acoustic field describing the shape of the focal point i.e. the spatial energy distribution induced by a treatment point;

FIG. 5 illustrates the architecture of a generator 256 generating electric signals;

FIGS. 6a to 6c show temperature maps obtained by electronically moving the focal point over four points;

FIG. 7 shows a succession of temperature maps obtained by electronically moving the focal point along two successive spirals;

FIGS. 8a to 8c illustrate the Fourier transform of the shape of the focal point i.e. of the spatial energy distribution induced by a treatment point in three orthogonal planes, plane k_{z}k_{y}, plane kₓk_{z}, and plane kₓk_{y} respectively;

FIG. 9 illustrates the excess energy produced compared with the required energy, due to overlapping of focusing points;

FIGS. 10a to 10c illustrate the first three iterations of the detection of maximum algorithm;

FIG. 11 illustrates the energy produced compared with required energy, obtained with the three first iterations of the detection of maximum algorithm;

FIGS. 12 to 15 illustrate the application of the detection of maximum algorithm, establishing a density of points (FIGS. 14i, 14ii and 14iii) and the energy produced (FIGS. 15i, 15ii and 15iii) by this density of points with a cubic distribution of required energy (FIGS. 13i, 13ii and 13iii) and a given shape of focal point (FIGS. 12i, 12ii and 12iii); the indices i, ii and iii characterizing the different figures indicate that the illustration is given along a transverse plane, the coronal plane and the sagittal plane respectively;

FIGS. 16a to 16c show the point density established by the detection of maximum algorithm in three central slices, with a cubic distribution of required energy;

FIG. 17 shows the trajectory of the 12 points taken from the point density shown FIGS. 16a to 16c;

FIGS. 18a to 18b illustrate the temperature time and space regimen, shifted by 5 s/mm for off-centre points;

FIGS. 19a and 19b show two thermal maps obtained during the temperature monitoring of a segment 11 mm in width;

FIG. 19c shows a thermal dose map obtained after temperature control of a segment 11 mm in width;

FIG. 20 shows the maximum, mean and minimum temperature over time for the 11 voxels controlled as in FIGS. 19a and 19b, compared with the set temperature;

FIGS. 21a and 21b illustrate the spatial temperature distribution along axes X and Z respectively, at the start, in the middle and at the end of the temperature rise conducted as in FIGS. 19a and 19b;

FIGS. 22a and 22b illustrate the spatial temperature distribution along axes X and Z respectively, at 4 moments during the heating temperature hold at 15° C. in FIGS. 19a and 19b;

FIGS. 23a and 23b show two thermal maps obtained during temperature control of a segment 11 mm in width, with shifting of the temperature regimen for off-centre points;

FIG. 23c shows a thermal dose map obtained after temperature control of a segment 11 mm in width, with shifting of the temperature regimen for off-centre points;

FIG. 24 shows the maximum, mean and minimum temperature over time for the 11 voxels controlled as in FIGS. 23a and 23b, compared with the set temperature;

FIGS. 25a and 25b illustrate the spatial temperature distribution along axes X and Z respectively at 5 different times during the temperature rise in FIGS. 23a and 23b;

FIGS. 26a and 26b illustrate the spatial temperature distribution along axes X and Z respectively, at 4 times during the heating temperature hold at 15° C. as in FIGS. 23a and 23b;

FIG. 27 shows the width at mid-height, i.e. 7.5° C., during the heating in FIGS. 19a and 19b and 23a and 23b obtained with a temperature regimen that is synchronized or shifted at each point;

FIGS. 28 and 29 show thermal maps at the middle and end respectively of temperature rise, and

FIG. 30 show the thermal dose maps obtained after temperature control on a cubic volume of 8×8×12 mm³; the indices i, ii and iii characterizing the different figures indicate that the illustration is given as per slice 3, slice 4 and slice 5 respectively;

FIG. 31 shows the maximum, mean and minimum temperature over time for the 147 voxels controlled as in FIGS. 28 and 29, compared with the set temperature;

FIGS. 32 and 33 show the spatial temperature distribution along axes X and Z in three slices, at the start, in the middle and at the end of the heating temperature rise in FIGS. 28 and 29; the indices i, ii and iii characterizing the different figures indicate that the illustration is given as per slice 3, slice 4 and slice 5 respectively;

FIG. 34 shows the temperature along axis Y at the start, in the middle and at the end of the heating temperature rise in FIGS. 28 and 29;

FIGS. 35 and 36 show thermal maps at the middle and at the end of the temperature rise respectively, and

FIG. 37 show the thermal dose maps obtained after temperature control of an elliptic volume 8×8×12 mm³; the indices i, ii and iii characterizing the different figures indicate that the illustration is given as per slice 3, slice 4 and slice 5 respectively;

FIG. 38 shows the maximum, mean and minimum temperature over time for the 87 voxels controlled as in FIGS. 35 and 36, compared with the set temperature;

FIGS. 39 and 40 show the spatial temperature distribution along axes X and Z in three slices, at the start, in the middle and at the end of the heating temperature rise in FIGS. 35 and 36; the indices i, ii and iii characterizing the different figures indicate that the illustration is given as per slice 3, slice 4 and slice 5 respectively;

FIG. 41 shows the temperature along axis Y at the start, in the middle and at the end of the heating temperature rise in FIGS. 35 and 36.

### DETAILED DESCRIPTION OF THE INVENTION

### Description of the Equipment

Global Description

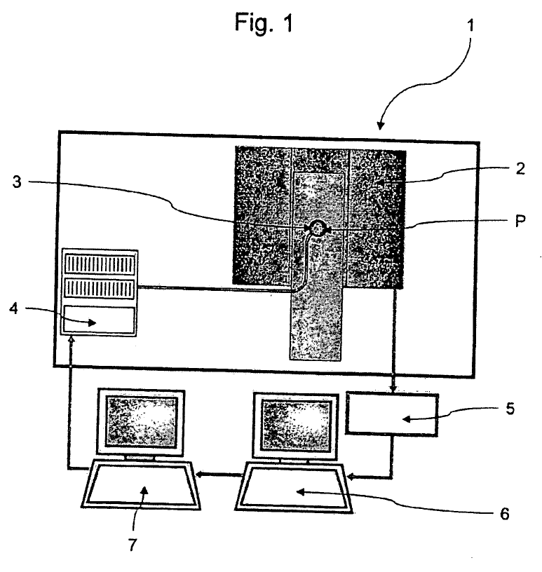

FIG. 1 shows a device to treat biological tissues comprising means to measure a target region of a biological tissue to be treated, to provide a measurement signal of the target region intended to characterize the target region (its movement for example). It is possible to use MRI imaging means 2 for example as measuring means, intended to provide images of the target region of the biological tissue to be treated. 1.5 Tesla MRI imaging apparatus may be used for example, allowing the simultaneous providing of 3D anatomical maps and temperature maps of the patient region of interest, with a spatial resolution in the order of one millimetre, accuracy in the order of 0.5° C. and temporal resolution in the order of one second.

The measurements acquired inside the magnet are converted to an image by the MRI reconstructor 5, which performs a Fourier transform and filtering before displaying this image on the acquisition unit 6.

Temperature mapping including the area to be heated is carried out by MRI. This data is transferred in real time by a fast rate network connection of the MRI acquisition unit 6 towards control means 7 of the device in the form of a monitoring unit. This control means 7 is particularly dedicated to the monitoring of temperature maps and guiding of the ultrasound system.

On the basis of these data the control means 7, using a computer programme for example, evaluates the position and intensity of the next focusing points on the basis of the anatomical and thermal maps.

The coordinates and the power of the next focal points are transmitted to energy generating means (3,4). More precisely, these data are transmitted to a 256-path signal generator 4, via optical fibres in a few milliseconds. This generator 4 produces and amplifies the ultrasound electric signals that are phase-shifted so that the matrix transducer 3 connected thereto emits an ultrasound wave focused on the chosen focal point P. The rise in temperature induced inside the focusing point therefore allows the thermal dose to be obtained that is needed to achieve necrosis.

Electronic Shifting of the Focal Point

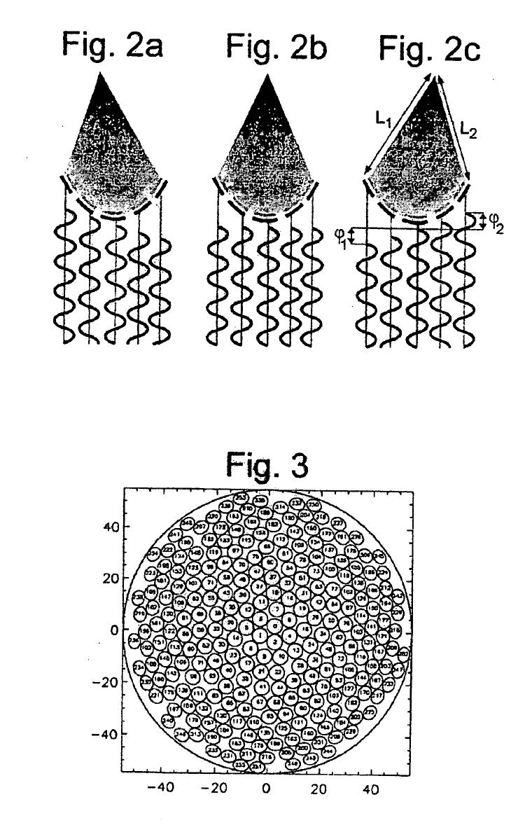

The principle of electronic shifting of the focal point consists of adjusting the phase of the electric signals so as to set up constructive interference at the desired position of the waves, derived from each of the elements. FIGS. 2a to 2c show an example of phase-shifting of the electric signals, inducing electronic shifting of the focusing point.

In the direction of propagation of the ultrasound wave, the phase varies by 2π for a wavelength λ. On this account, the phase Φₙ of the electric signal of element number n relative to element number 0 is calculated using the following phase law:

$${Φ}_{n} = 2 π \frac{{L}_{n} - {L}_{0}}{λ} \qquad ( Equation 1 )$$

In this equation, the length Lₙ corresponds to the distance between the centre of element n of coordinates (xₙ, yₙ, zₙ) and the desired focusing point of coordinates (x_{F}, y_{F}, z_{F}). This length is calculated directly using Pythagoras' formula:

Lₙ=\sqrt{(Xₙ−X_{F})+(Yₙ−Y_{F})²+(Zₙ−Z_{F})²} (Equation 2)

This manner of calculating phase shifting is approximate since it assumes the point element, but it gives the same result as obtained using a more complete analysis of the acoustic field. Calculation of the phase of the electric signals using the equation (Equation 1) is very accurate and very swift since it solely requires calculation of a square root for length Lₙ. During control of spatial temperature, since the calculation of phase shifts is made very frequently, this method is always the one used.

Compared with mechanized shifting of the transducer, electronic shifting of the focal point is most useful for controlling temperature spatially, since it allows travel over a large number of points to be heated per second. In addition, since the transducer remains immobile, this movement of the focal point does not create any imaging artefact related to change in magnetic susceptibility. Electronic moving of the focusing point does not have any limitations regarding speed but, on the other hand, it is subject to another type of limitation:
there is no speed limitation since the position of the focal point is redefined at each instant by the phase of the electric signals applied to the transducer. Only the electric signal generator used requires a certain time for data transfer before switching all the paths to the new desired value. Therefore, the minimum time between each change in the position of the focal point is 60 ms.
Secondly, the focusing effect can only be produced where the composite piezo elements emit a signal. This region is defined by the effect of diffraction which is dependent on the size of the elements, the frequency used, the focal length of the transducer and to a lesser extent its shape. For the matrix transducer described below, the shifting of the focal point is confined within an ellipsoid of size 15 mm×15 mm×28 mm.

Since there is no longer any real constraint regarding speed of movement, the spatial Proportional-Integral-Derivative (PID) control can be optimized. Helpful reference may be made to French patent application n^{o} 99-11418 filed on 13 Sep. 1999 titled “Thermal treatment assembly for biological tissues and method to implement this assembly” for a more detailed description of this type of temperature regulation system, which is incorporated herein by reference. On the other hand, the main drawback for electronic shifting is its small amplitude of movement which limits heating to small volumes.

Description of the Matrix Transducer

For this application, an ultrasound matrix transducer operating at 1.5 Mhz was developed in collaboration with the company Imasonic. This transducer with focal length of 80 mm and radius aperture of 55 mm forms an angle of aperture of 86°. The elements chosen for this transducer have a radius of 2.9 mm so as to have freedom of positioning of the focal point ranging from −16 mm to 13 mm along the axis of revolution and ±7.5 mm along the two orthogonal axes. The distribution of the elements on the surface of the transducer is compact asymmetric, so as to minimize secondary lobes when moving the focal point.

FIG. 3 shows the configuration of the elements chosen for manufacture of the transducer. This allows the focusing point to be moved over the above-described range with secondary lobes of less than 7%.

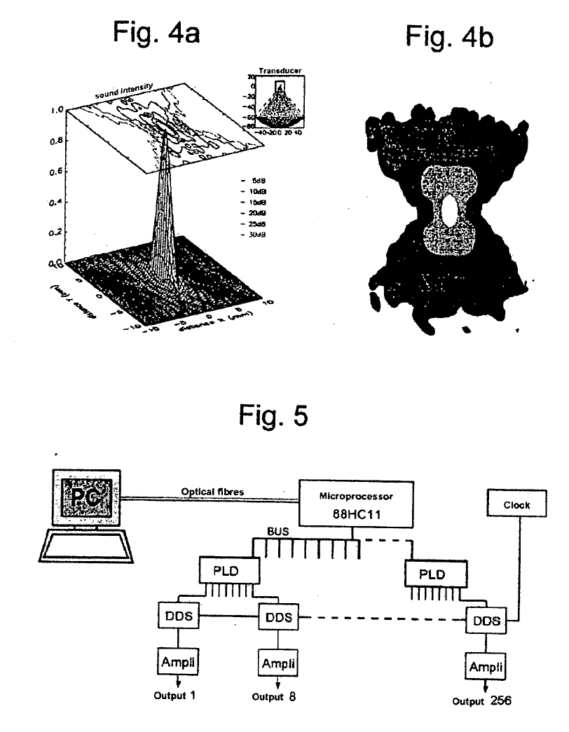

FIGS. 4a and 4b illustrate a simulation of the acoustic field produced by this geometry of the transducer on a 20×20 mm² window. For this transducer with aperture angle of 80°, the size of the focal point is 0.76×0.76×3.47 mm³. This transducer geometry provides fairly generic use, since an acoustic window with small aperture angle is sufficient.

For full compatibility with 1.5 T MRI, each of the parts used for the manufacture of this transducer and the associated cables and connectors are designed to interfere the least possible with the magnetic field. For this purpose the materials chosen, copper, tin, lead, ceramic, resin, silicon plastic have a magnetic susceptibility between that of water and air. With these materials, the transducer does not induce any distortion on MRI images. Additionally, the contours of this transducer are distinctly apparent in MRI images, which enables its position to be easily identified and hence the position of the focal point produced.

Description of the Multi-Path Generator

The electronic architecture of the 256-path signal generator allowing the use of a matrix transducer is illustrated FIG. 5.

A MOXA C104H card included in the PC increases the speed of the serial port from 115200 Kb/s to 460800 Kb/s. By transmitting only essential information, frequency, phases and amplitudes, all the data for redefining sinusoids are transmitted in 22 ms.

Also, the optical fibres, in addition to permitting fast bit rates over long distances, provide excellent protection against electromagnetic interference. They allow the data to pass through the Faraday Cage of the MRI without bringing any outside disturbance. In this way, the connection between the monitoring unit outside the MRI room and the generator inside the MRI room is ensured without interfering with the operation of the MRI apparatus.

Additionally, the generator is developed to operate inside a MRI room. The cards were designed to generate low electromagnetic radiation. Therefore its installation merely requires the installation of two optical fibres so that each cable can pass through the Faraday Cage, instead of 256 wall filtering boxes as was previously the case.

This generator is managed by a 68HC11 microprocessor. It distributes data to the Programmable Logical Device (PLD) on the 32 cards which each generate 8 sinusoids. The PLDs then address the data on phase and frequency to the Direct Digital Synthesis (DDS) generators, and power to the amplifiers. The DDSs with original design for phase and frequency modulation ensure fast accurate signal switching. This represents a major modification compared with other generators of ultrasound signals, which with lag lines, do not exceed a phase accuracy of more than ±5°. The amplitudes of the sinusoids are then adjusted by variable gain amplifiers. The use of a clock common to all DDSs ensures perfectly synchronous use of all output stages. In other words, by means of this common clock, the phase of each of the sinusoids is referenced in the same way.

Signal definition is made by transmission of a file containing the data in this order:
4 optional octets to encode the frequency common to all the outputs, having a value of:

$$F = 24 MHz \times \frac{Nbr encoded over 32 bits}{{2}^{32}}$$

The frequency is therefore defined between 0 and 24 Mhz to an accuracy of ±3 Mhz.
512 octets, over 2 octets, define the respective phase of each signal as per the formula:

$$φ = {360}^{°} \times \frac{Nbr encoded over 16 bits}{{2}^{12}}$$

This allows all the phases to be adjusted between 0 and 360° to an accuracy of ±0.04°.
256 octets are used to adjust the output power of each path as per the equation:

$$P = 4 W \times \frac{Nbr encoded over 8 bits}{{2}^{8}}$$

The output power can therefore vary up to 4 W to an accuracy of ±8 mW.

The format of the communication protocol is X-MODEM in half-duplex connected mode. Each transmitted packet of 128 octets is acknowledged by the receiver which controls receipt with a Cycle-Redundancy Check (CRC) encoded over 1 octet. In this manner, the computer can transmit a file defining the frequency, phases or amplitudes every 22 ms using a secure protocol.

This data is then stored on a memory card in the vicinity of the 68HC11 microprocessor, then redistributed to each of the 32 PLD logic cards. When all the logic circuits have received their information, a switch signal is sent for all the cards simultaneously. From this moment onwards, each logic circuit modifies the frequency, phase and amplitude of all the sinusoids at the same time. This allows transitory states to be avoided in which the ultrasound beam is partly defocused and hence detrimental to patient safety.

Although bound by limitations laid down by the programme implemented in the microprocessor, the minimal time for modifying the status of all outputs is limited to 60 ms.

To summarize, this generator is designed so as to associate:

Rapidity of signal redefinition

Simultaneous changing of signals

Low electromagnetic radiation

Accurate defining of output sinusoids

Secure data exchange

Portability

Example of Electronic Shifting of the Focal Point

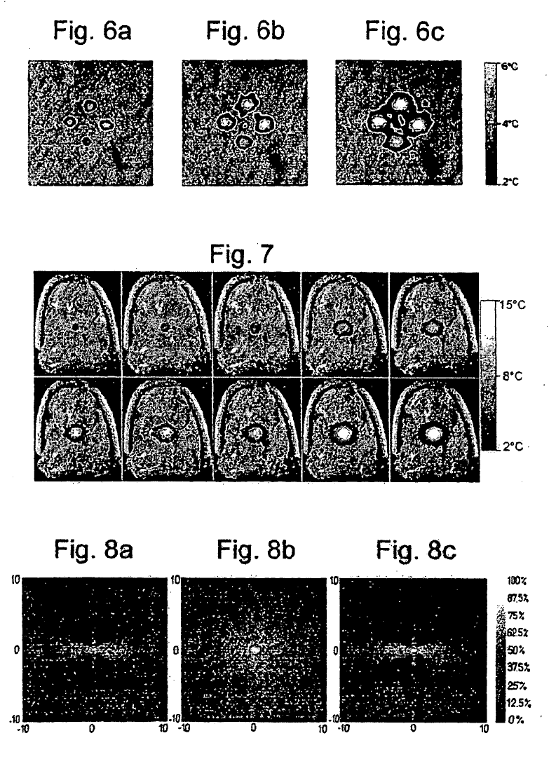

To test the principle of electronic shifting of the focal point and proper functioning of the matrix transducer, the focusing point was moved 4 points around a square with sides of 8 mm. This trajectory was repeated cyclically every 0.5 s by changing the position of the focusing point every 125 ms with a constant electric power of 200 W. By simultaneously conducting 5 slices of 4 mm (echo time 18 ms) every 3.9 s, the heating observed in FIGS. 6a to 6b appears to derive from 4 focusing points simultaneously.

This method of moving the focal point is very rapid since there is no longer any maximum displacing speed but just a minimum electronic signal switching time of 60 ms with the generator used. This technique is also very accurate. The phase definition accuracy of the signals produced is a 2¹² fraction of a period corresponding to a wavelength of 1 mm. Additionally, phase errors between the 256 signals average each other out. The accuracy of theoretical positioning of the focal point is 1 mm/(256×2¹²)=1 nm. However, this result remains difficult to verify, even by simulations which would require very small meshing and very high numerical accuracy.

The type of possible trajectory can be chosen in any manner provided that each point is not distanced more than 7.4 mm away from its central point. FIG. 7 shows all the thermal maps obtained during two successive spiral heatings. Each spiral of diameter 10 mm consists of 96 points and lasts 20 s. The sequence used is the same as previously at the rate of one acquisition every 3.9 s.

The power applied to each of the points is a constant electric power of 50 W that is intensity compensated in relation to amplitude of movement. In this way, the heating obtained is homogeneous for as long as the tissue has no heterogeneity, as appears to be the case in this experiment. Power or point density may possibly be controlled by the 2D temperature control algorithm described further on. However, since the focal point can be moved electronically in all directions of the space, it is preferable to use three-dimensional (3D) temperature control.

### Volume Temperature Control

### 3D Energy Distribution Using the PID Spatial Algorithm

Spatial temperature control is based on an extension of the differential equation of PID automation at each point of the space:

$$\frac{\partial ξ}{\partial t} ( \vec{r} ) + q ξ ( \vec{r} ) + \frac{{q}^{2}}{4} {\int }_{0}^{t} ξ ( \vec{r} ) = 0 \qquad ( Equation 3 )$$

In this equation, the spatial and temporal variable ξ is the difference between the set temperature Tc and the measured temperature T at a time t at each of the points \vec{r} of the space:

ξ_{(\vec{r},t)}=T_{C(\vec{r},t)}−T_{(\vec{r},t)} (Equation 4)

The principle of spatial and temporal temperature control treats each of the points of the space individually. Therefore the set temperature of each of the heated points can be defined independently of each other. However, convergence towards the desired temperature is not always guaranteed since it is difficult to heat one point on its own. In the vicinity of the focal point, especially along the wave propagation axis, a rise in temperature always subsists. Also, the effect of thermal diffusion tends to propagate heating from one point to an adjacent point. For these reasons, temperature control can be made provided that the spatial resolution of the set temperature does not exceed the spatial resolution of the focal point, and provided the temperature gradient between two adjacent points is not too high relative to the coefficient of diffusion. The thermal transfer equation used to anticipate tissue behaviour and the interaction between the different heated points is as follows:

$$\frac{\partial T}{\partial t} ( \vec{r} ) = D \cdot {∇}^{2} T ( \vec{r} ) + α \cdot P ( \vec{r} ) \qquad ( Equation 5 )$$

Therefore, the PID differential equation is balanced when the applied power is chosen so that:

$$P ( \vec{r} ) = \frac{1}{α} [ \begin{matrix}\frac{\partial {T}_{C}}{\partial t} ( \vec{r} ) - D \cdot {∇}^{2} T ( \vec{r} ) + q ( {T}_{C} ( \vec{r} ) - T ( \vec{r} ) ) + \\ \frac{{q}^{2}}{4} \int ( {T}_{C} ( \vec{r} ) - T ( \vec{r} ) )\end{matrix} ] \qquad ( Equation 6 )$$

It is very difficult to apply a spatial distribution of power i.e. to heat several points simultaneously. This can be achieved using a matrix transducer which simultaneously focuses several points. Such use of matrix transducers is limited however, and very dangerous since it induces numerous adverse secondary lobes. A result of better quality is obtained more simply by successively focusing each of the points.

Irrespective of the manner in which the focal point is moved, whether electronic or mechanical, it is necessary to take into account the energy to be deposited at each of the points, rather than the spatial distribution of the power to be applied:

$$E ( \vec{r} ) = \frac{{t}_{A}}{α} [ \begin{matrix}\frac{\partial {T}_{C}}{\partial t} ( \vec{r} ) - D \cdot {∇}^{2} T ( \vec{r} ) + q ( {T}_{C} ( \vec{r} ) - T ( \vec{r} ) ) + \\ \frac{{q}^{2}}{4} {\int }_{t} ( {T}_{C} ( \vec{r} ) - T ( \vec{r} ) )\end{matrix} ] \qquad ( Equation 7 )$$

The time interval t_{A} is the time needed to cover all the points to be heated. This time period also defines the frequency at which PID control is calculated. To guarantee the stability of the counter-reaction, the response time of the system t_{R} (equal to 2/q) must be greater than the control time t_{A}. In general the response time is chosen to be 3 times the control time.

To implement the PID controlled regulation, the proportion, integral and derivative term is calculated in the same manner as for point control. Similarly, anticipation of behaviour can be calculated using a Fast Fourier Transform (FFT) or discretely. Since the spatial PID control is based on the same equations as the point PID control, it offers the same stability with respect to the tissue parameters used.

To better control the heated volume, it is preferable to conduct 3D spatial temperature control using the equation (Equation 7) over the entire volume. This does however require the ability to deposit the 3D spatial energy distribution thus calculated. This is fairly complex having regard to the large number of voxels contained in the volume, and especially to the overlap effect previously described.

Method for 3D Energy Deposit

The generic equation (Equation 7) can be used to calculate required energy in all cases, to obtain the desired temperature in three dimensions. Although the matrix transducer associated with a multi-path signal generator is a very rapid tool, it is not always easy to produce this energy.

Having regard to material limitations, some spatial energy distributions are not feasible. For example, calculation of the PID equation (Equation 7) at a point largely hotter than its set temperature generally leads to a negative energy value. Although cryotherapy tools exist, ultrasound transducers do not allow extraction of a quantity of energy. Therefore PID control is no longer feasible at this point. The solution is then to wait until the effect of diffusion or perfusion cools the tissue sufficiently.

Similarly, if the required spatial energy distribution is a Dirac of 1×1×1 mm³, it is not feasible with a focusing point of 0.76×0.76×3.47 mm³ which is three times longer. It is therefore necessary to take into account the spatial energy distribution E^{1pt} induced by a focusing point. If moving of the focal point can be likened to a translation, the energy deposited by a trajectory E^{Traj} is quickly calculated in the Fourier space using the following formula:

\tilde{E}^{Traj}=\tilde{Γ}×\tilde{E}^{1pt} (Equation 8)

In this expression, the trajectory function Γ is the spatial distribution of point density. Up until now, for 2D temperature control, the energy produced by a trajectory was considered similar to point density since in the coronal plane the focal point corresponded to one voxel. This rule is verified for as long as the width of the voxel acquired by MRI in the coronal plane does not exceed 0.76 mm. For a three-dimensional study, this approximation is never verified owing to the overlap effect of the ultrasound propagation cone. This effect can induce heating over several centimetres along the axis of propagation, as shown in the example of spiral trajectories. The whole of all energy distributions produced is therefore limited by this overlap phenomenon which is dependent on the spatial distribution of the acoustic field. According to the equation (Equation 8), this whole is directly related to the transform of the shape of the focal point shown FIGS. 8a to 8c.

Since the transform of E^{1pt} is zero for the majority of points in which k_{Y} is non-zero, the same applies to the transform of E^{Traj}. On the other hand, E^{1pt} completely covers the plane k_{X}k_{Z}. In other words, the whole of all feasible energy distributions cannot comprise a detail in direction Y but can contain details in directions X and Z. As a general rule, in the Fourier space, feasible energy distributions are zero where the transform of E^{1pt} is zero. Additionally, there must be a positive trajectory function verifying the equation (Equation 8) since point density cannot be negative.

To conduct temperature control, a reverse operation must be applied to the operation used to simulate the acoustic field. Instead of calculating energy distribution using the trajectory, the trajectory must be deduced from an energy distribution. Having regard to the constraints previously mentioned, this problem can be solved using a deconvolution as described by the following equation:

\tilde{Γ}=\tilde{E}^{Traj}/\tilde{E}^{1pt} if \tilde{E}^{1pt}≠0

\tilde{Γ}=0 if \tilde{E}^{1pt}=0 (Equation 9)

In the Fourier space, if E^{Traj} is non-zero, then Γ corresponds to the ratio of E^{Traj} over E^{1pt}, and if E^{Traj} is zero then Γ is chosen to be zero to minimize the energy deposited by this trajectory. Then, in the real space, only the positive values of the trajectory function are maintained.

Unfortunately, this solution minimizing the difference between the required energy and the energy produced over the entire observation window, tends to reduce the energy on the target volume to minimize the energy deposited outside thereof. Depending on the size of the observation window, and hence on the number of voxels outside the target volume, under-evaluation of the energy to be deposited is of greater or lesser extent. Also the product of convolution by a positive E^{1pt} function is a low-pass filter. Deconvolution by E^{1pt} produces the reverse effect, which enhances contours and amplifies noise. Since the trajectory function obtained using the deconvolution method depends on the size of the observation window and has a signal-to-noise ratio lower than that of calculated energy distribution, it is necessary to use another solution to maintain the stability of PID control.

Detection of Maximum Algorithm

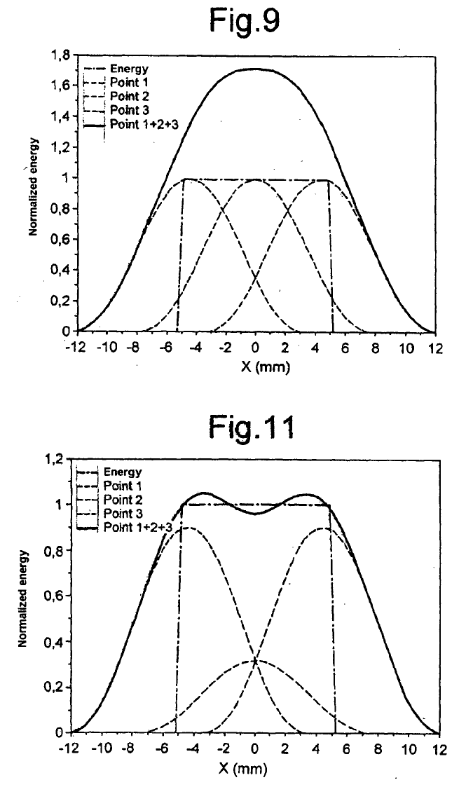

The trivial solution used for 2D temperature control, which consists of choosing the trajectory function to be equal to the spatial distribution of required energy, is efficient for as long as the overlap effect is negligible. For illustration thereof, FIG. 9 shows three forms of focal points grouped together to form a square spatial distribution.

In this graph, the red curve represents the required energy and the black curve represents the energy produced by the translated focusing points. The secondary lobes of each focal point, over a longer distance, add themselves to the other focusing points whose centres have already reached the required energy value. Therefore the energy produced in this example exceeds the desired level by 70%. The shape of the focal point used here corresponds to the intensity measured along the propagation axis of the wave, magnified by a factor of 2 to enhance overlap. In practice, since superimposition is made in three dimensions, this excess attains much higher values.

One less radical solution consists of deducing each point by iteration, by observing the influence of the other points. The purpose of this algorithm is to find the trajectory with lowest energy to reach the required energy level at every point in the space.

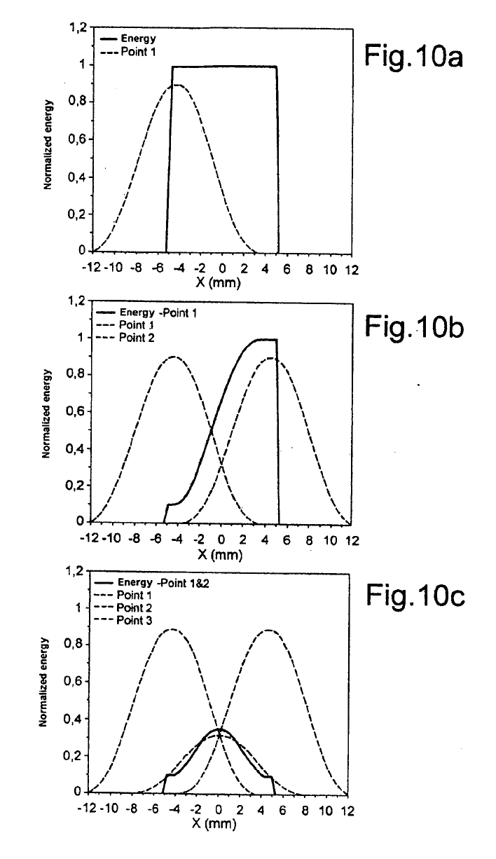

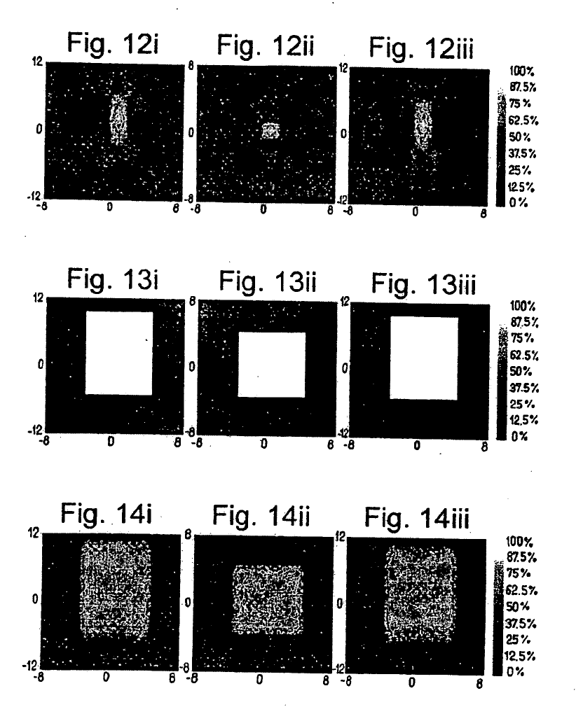

The principle of focusing is to concentrate all emitted energy onto one point. On this account, for a required point energy, the best yield is obtained by focusing on this point. Similarly, for any spatial distribution of the required energy, it is optimal to focus on the point at which required energy is maximum. However the focusing energy applied to this point depends on the focusing points made around it. So as not to reproduce the error illustrated FIG. 9, the energy applied to this point must be less than the required energy level. Arbitrarily, the energy to be applied to this point is then chosen to be equal to a percentage R of the value of the required energy. Once this focusing point to be applied has been taken into account, the algorithm is iterated searching the point corresponding to the maximum distribution of required energy subtracted by the energy distribution(s) arising from the focusing point(s) previously deduced. FIGS. 10a to 10c show the first three iterations of this algorithm applied to a square function, with a percentage R of 90%.

With Eⁱ(\vec{r}) being the difference between required energy and the energy produced by the i focusing points previously deduced, and Γⁱ(\vec{r}) being the corresponding trajectory function, this algorithm is mathematically written in the form of the following sequence:

$${E}^{0} ( \vec{r} ) = {E}^{REQUIRED} ( \vec{r} ) and {Γ}^{0} ( \vec{r} ) = 0 \\ \vert \begin{matrix}let {\vec{r}}_{n} such that {E}^{n} ( {\vec{r}}_{n} ) = max ( {E}^{n} ( \vec{r} ) ) \\ {E}^{n + 1} ( \vec{r} ) = {E}^{n} ( \vec{r} ) - R \cdot {E}^{n} ( {\vec{r}}_{n} ) \cdot {E}^{1 pt} ( \vec{r} - {\vec{r}}_{n} ) \\ {Γ}^{n + 1} ( \vec{r} ) = {Γ}^{n} ( \vec{r} ) + R \cdot {E}^{n} ( {\vec{r}}_{n} ) \cdot δ ( \vec{r} - {\vec{r}}_{n} )\end{matrix} \qquad ( Equation 10 )$$

In this equation, the value of the Dirac function δ is 0 everywhere except at the origin where its value is 1. Since temperature control is conducted over a finite number of voxels N, after N iterations the maximum remaining energy Eⁿ decreases at least by a factor 1−R:

max(E^{n+N}(\vec{r}))≦(1−R)·max(Eⁿ(\vec{r})) (Equation 11)

This inequality certifies a geometric convergence such that the maximum remaining energy converges towards 0. The maximum focal point energy to be applied is chosen to be a percentage of the remaining energy, instead of a constant pitch, to obtain this geometric convergence rather than an arithmetic convergence. Also the convergence of the sequence of the trajectory function Γⁿ is guaranteed since each of its points represents an increasing sequence limited by the trivial function given FIG. 9.

At convergence, the trajectory function Γ obtained corresponds to that of the smallest energy reaching the required energy at each of the points. If the required energy distribution is not part of feasible functions (such as the square root) the energy produced by this trajectory exceeds the desired energy level at some points (on the outside edges of the square). But at least necrosis is obtained on all the voxels desired to have lesser energy.

FIG. 11 shows the energy produced by the three focusing points shown FIG. 10. Convergence is not achieved since the central point does not reach the required energy. The following iteration would consist of increasing the value of the central point. In all cases convergence is never attained since at each step there always remains a percentage 1−R of the maximum. On this account, it is essential to use a stop criterion. The one chosen is the comparison of the last maximum with a percentage of the initial maximum, which makes it possible to define the obtained accuracy of produced energy.

Also, the factor R defines the rate of convergence of the algorithm according to the equation (Equation 11). The closer R is to 1, the faster convergence, but also the greater the risk of obtaining a value that is too high to estimate the energy to be deposited at the detected maximum point. To avoid such unnecessary overstepping of produced energy, 1−R can be chosen to be equal to or greater than the sum of the secondary lobes of the related focusing points normalized to one. Since this result depends on the spacing of the focusing points, and hence on the resolution of MRI imaging, it is simpler to choose a relatively low value for R. This choice is not very restricting, since even with a convergence factor of 10% for an accuracy of 2%, the calculation time does not exceed 10 ms for a 1 GHz processor. This does however require optimizing the code, by searching the maximum and calculating the remaining energy solely on the N voxels which are controlled. If not, the code is slowed by a factor of 100 or more, depending on the size of the observation window.

The trajectory function obtained is identical irrespective of the chosen R factor, provided that it does not exceed a certain value. The only ambiguity lies in the choice of the maximum in the event of an equal value, which is the case for the first iteration applied to a uniform required energy such as the square previously described. In practice this odd case does not occur frequently, and the choice of one maximum rather than another does not have any real consequence since they both require energy deposition. The solution chosen is the first maximum encountered since the regions located on the periphery of the heated volume require a higher point density.

FIGS. 12 to 15 illustrate the application of this detection of maximum algorithm to a cubic 3D volume 7×7×9 mm³ with a factor R of 10% and an accuracy of 5%. Similar to the MRI, the resolution used is 6 slices consisting of voxels 1×1×4 mm³. The point density obtained is chiefly located in the corners of the cubes, then on the edges and preferably on the upper and lower sides. In short, it is a hollow shape. In general, irrespective of the form of the required energy, most point density lies on its periphery.

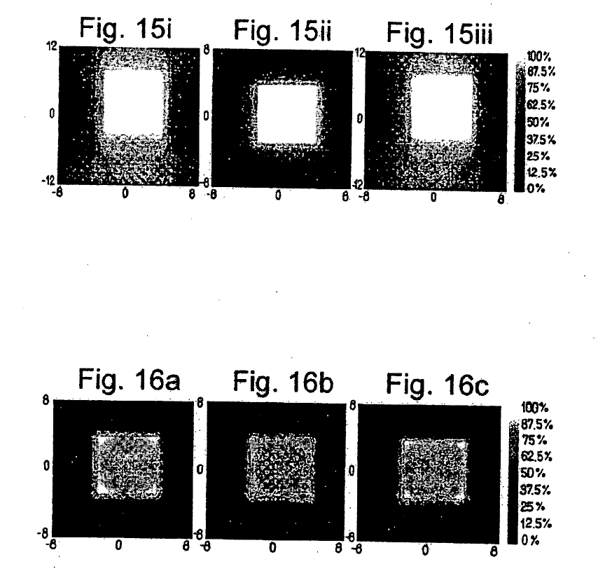

To verify the accuracy of the result, the energy produced calculated using the equation (Equation 8) with this point density, is also reproduced in FIGS. 15i, 15ii and 15iii. As expected, it corresponds to the required energy to within 5% over the entirety of the cube. On the other hand, energy is inevitably produced on the outside of the cube. It especially lies along axis Y where the cones of ultrasound propagation overlap.

The detection of maximum algorithm can be used to define point density with precision, swiftness and stability. Since the high frequencies of noise have non-feasible high gradients (especially along axis Y) the signal-to-noise ratio of the trajectory function obtained is equal to or greater than that of the required energy distribution. In addition, in the case when this distribution is not feasible, the produced energy at some points exceeds the required energy to reach the desired temperature at all points with minimum energy. All these advantages fully correspond to the therapeutic need, the only drawback being the mean overstepping of required energy, which increases with the increase in noise level and the number of controlled voxels.

Extraction of a Trajectory

Once the spatial distribution of point density has been calculated using the detection of maximum algorithm, a trajectory must be extracted which takes into account all the material limitations set forth above.

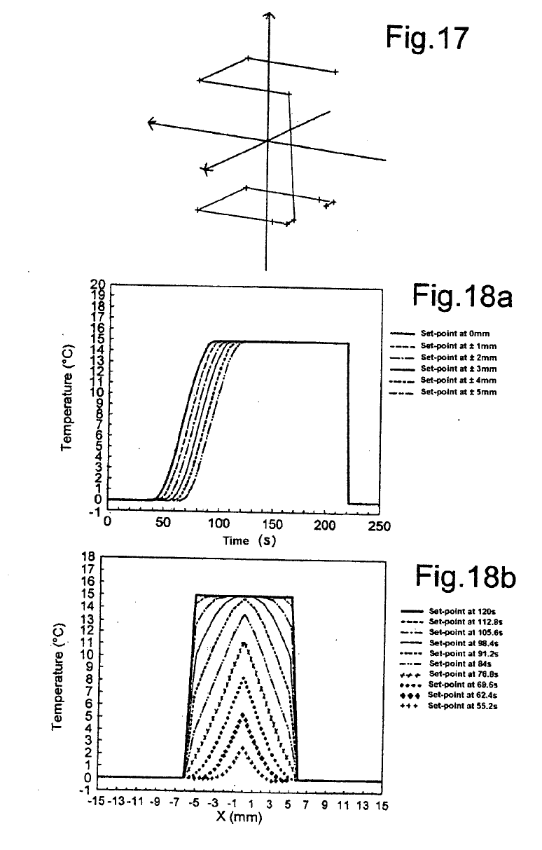

The trajectory function obtained previously (see FIGS. 16a to 16c) consists of 3 slices in which a square of 7×7 voxels is non-zero. Ideally a trajectory of 147 points should be obtained so as to produce the required energy to within 5%. However, the time length of the trajectory is chosen to be equal to the time length of a dynamic, so that it is possible to produce the counter-reaction as frequently as possible. Therefore the control time is chosen to be equal to the acquisition time of a dynamic i.e. 2.4 s in this case. Having regard to the fact that the minimum switch time of the generator is 60 ms, this only allows a maximum of 40 points to be treated during this time interval. Additionally, a trajectory must be used whose time length is shorter than the acquisition time of a dynamic since the image transmission and processing time is not perfectly regular. It is therefore most helpful to keep a flexibility margin of a factor of 2 regarding point duration. This freedom allows points requiring very high energy levels to be treated over a longer time with moderate power, rather than short-time heating with very high power. High levels of acoustic power, even very short, are effectively sufficient to trigger a cavitation effect which modifies tissue behaviour, and is not the effect sought after here. To conclude, only a trajectory lasting 2.2 s with 12 points, as shown FIG. 17, is chosen from among the 147 points of the initial trajectory function.

The points chosen are those with the highest energy levels, the other points generally being covered by the following trajectory if judged necessary by the calculated counter-reaction. The temporal order of these points on the trajectory does not appear to have any significant effect on induced heating, taking into consideration the short time lengths used.

Once the position of the points and their energy level have been defined, it is possible to carry out this trajectory by adjusting the time length or power of each of its points. When this choice is possible, it is preferable to modify time length rather than power. It is much more cautious to use sonications of longer time length rather than stronger power, having regard to wear of the equipment as well as to patient safety. Evidently, the time length of a point must not exceed 2.2 s since the sum of all the points must be equal to the time length of the chosen trajectory. Similarly, a minimum limitation of 80 ms is imposed for each of the points since the switching time of all the generator signals is limited. Regarding the powers used, these are limited here to a maximum electric power of 200 W, but there is no lower limit. Evidently when a zero power is associated with one of the points of the trajectory, this is automatically removed from the list.

In short, all these sometimes contradictory constraints require the use of an algorithm to define the time length and power of each of the points. This algorithm is initialized by associating an identical power with each point, but of variable duration calculated so as to achieve the desired energy distribution. The power constant initially chosen is of no importance since it is subsequently modified.

The first step of the iteration loop consists of modifying the time length of all the points by one same factor so as to obtain the desired trajectory duration. The power of each of the points is divided by this same factor to maintain the desired energy at each thereof.

The purpose of the second step is to impose a minimum time upon all points whose duration is too short. For those points whose duration is lengthened, power is reduced to maintain initially defined energy.

The third and final step is to decrease the power of all the points exceeding the chosen maximum power. To maintain the predefined energy, the duration of the modified points is then increased.

Since these two latter steps increase the total time of the trajectory, the algorithm reiterates the calculation at the first step around ten times. This algorithm is performed very rapidly (in a time of less than one millisecond) since the number of treated points is low (12 points). However, in some cases, there is no trajectory which pays heed to these limitations with the desired energy. For example, if the total desired energy is greater than the product of maximum power times the duration of the trajectory, or if the number of points multiplied by the minimum time exceeds the duration of the trajectory. But on leaving the iteration loop, limitations regarding maximum power and minimum time are always verified. Only the duration of the trajectory can be longer than planned. In this case, the last points are not treated. Like the other points of the initial trajectory function, these points are treated during the following dynamic.

Transition from required energy to the point density function is limited by the shape of the focal point and hence of the transducer. Thereafter, the conversion of the trajectory function to a discretized trajectory is rather more limited by the signal generator used.

Time-Shifting of the Set Temperature

The methods described up until now allow the trajectory to be calculated in order to reach the desired temperature. All that is subsequently required is to choose a suitable set temperature to carry out the desired heating. Most often this temperature is temporally defined by a target temperature of 12° C. to 15° C. over a few minutes, so as to obtain the necessary thermal dose for tumour ablation. This set temperature can be chosen to be much lower for non-destructive use for example for gene expression or local depositing of medication. The transitory phase of temperature rise is obtained by a sinusoid half-period. This prevents creation of discontinuity of the set curve and its derivative, which rather benefits control stability.

Regarding the spatial distribution of the set temperature, it is generally chosen to be uniform over the entire tumour, and zero outside thereof, for the purpose of obtaining homogeneous treatment of the target region whilst best protecting adjacent tissues. Therefore, the temperature schedule is a square function whose amplitude varies according to the previously defined set time.

However, the temperature rise of an entire volume often requires a considerable quantity of energy, which may last one to two minutes taking into consideration material limitations and the size of the volume. However, during this time interval, heating propagates towards the outside by diffusion effect. One method to limit this effect consists of modifying the distribution of the set temperature so as to terminate the temperature rise at points located on the periphery of the volume, as with a spiral trajectory. For this purpose, the same set time is used for each of the points that is slightly shifted in proportion to their distance from the central point. In this manner, the central point is heated first. This heating then propagates to adjacent points at a rate defined by the operator. FIGS. 18a and 18b show an exemplary time-setting used subsequently to heat a segment of 11 voxels (from −5 mm to +5 mm) shifting this time-setting by 5 s for each millimetre.

Consequently, the time-settings translate one from another, except for the end of treatment which is synchronized for all points. The central points effectively cannot cool before the peripheral points. The spatial distribution of the temperature setting assumes a similar form to the time-setting over an interval of 25 s. If the temperature rise is linear over time, the space setting forms a straight line, and similarly if it is curved. Through effect of symmetry relative to the central point, the spatial temperature setting initially forms a triangle of increasing amplitude and width. Once the desired width and amplitude have been attained, this spatial setting becomes rounded and gradually forms the square function which allows subsequent homogeneous treatment.

### Experimental Results

To assess the efficiency of this temperature control method, several controls were conducted with the matrix transducer and 256-path generator previously described. The acquisition sequence used was always the same with 6 slices of 128×128 voxels of size 1×1×4 mm³. The echo time of the gradient echo sequence used was 18 ms and the repetition time 300 ms. Also, known image processing techniques were systematically applied to the results given below.

Linear Temperature Control

The first spatial control conducted covered a segment of 11 mm aligned along the X axis. The temperature regimen used was homogeneous heating at 15° C. for 120 s to obtain 3.5 times the thermal dose. The temperature rise was first conducted in synchronized manner for the 11 controlled voxels.

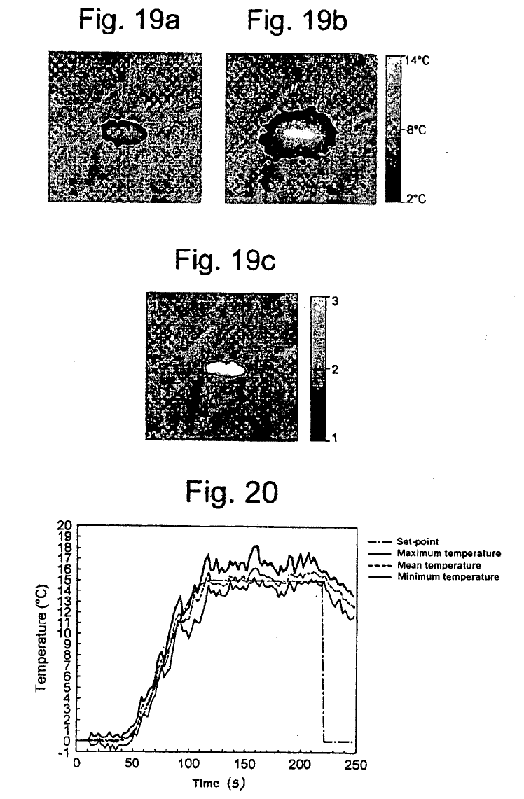

FIGS. 19a and 19b show two temperature maps acquired in the middle and at the end of the temperature rise. The heated form corresponds exactly to the one desired, i.e. a segment of width 11 mm with homogeneous temperature. As always, thermal diffusion propagates the temperature to adjacent tissue which slightly extends obtained necrosis which, as illustrated by the thermal dose map FIG. 19c, measures 12×4 mm² at the end of heating.

For a study that is more statistical than visual, FIG. 20 shows the minimum, mean and maximum temperature for the 11 voxels in which PID control was performed. All these points follow the set temperature to within a mean of 0.6° C. The difference between the minimum and maximum point is around 2° C. throughout the entire heating. Therefore all the controlled points induce the desired thermal dose as per the chosen temperature regimen.

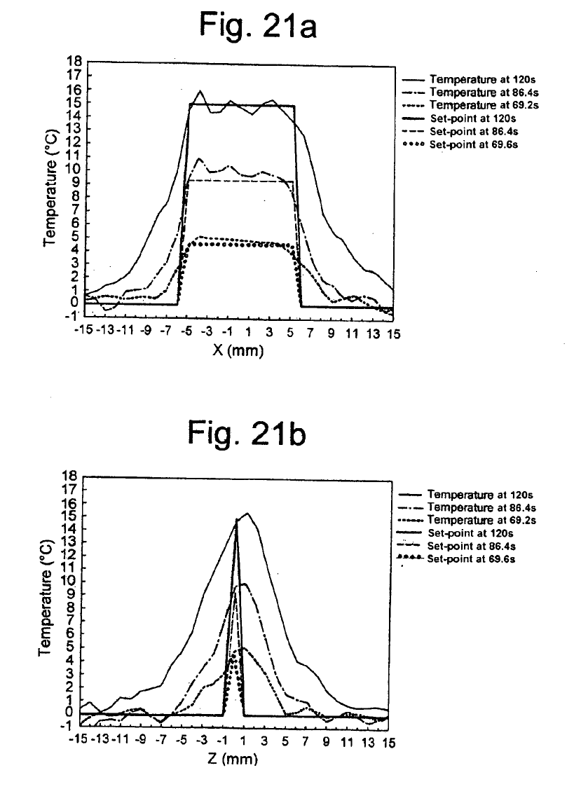

FIGS. 21a and 21b show temperature distribution at the start, in the middle and at the end of the temperature rise along axes X and Z. The temperature rise produced by the multiple focusing points forms homogeneous heating whose amplitude varies according to the previously chosen spatial and temporal settings. Solely the effect of thermal diffusion, which constantly opposes the establishing of a temperature gradient, prevents the production of a square function. Thermal diffusion, in all directions of the space, widens the heated area.

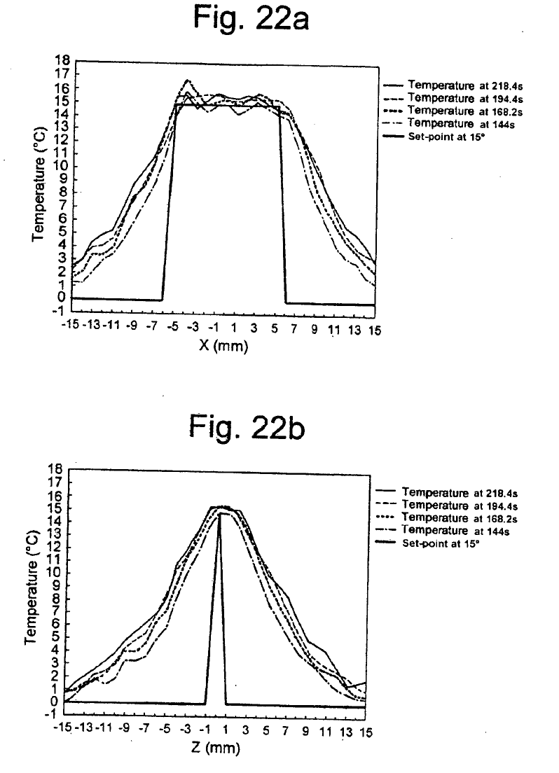

As shown in FIGS. 22a and 22b, once the set temperature of 15° C. is reached, this is held precisely up until the end of heating. The only difference between the different thermal maps is the gradual extension of the heated area through thermal diffusion.

Shifted Linear Temperature Control

The preceding experiment was reproduced, replacing the temperature regimen by the one shown FIGS. 18a and 18b in which the temperature rise is slightly deferred for those points distanced away from the centre. The purpose of this modification is to reduce temperature spread. So as to be comparable with the previous heating, this new temperature regimen was chosen as previously with a temperature rise over 80 s for all points. The maximum set temperature was 15° C. for 100 s to produce the same thermal dose on the peripheral points. The central points were slightly more necrotized, since they reached the maximum of 15° C. slightly earlier.

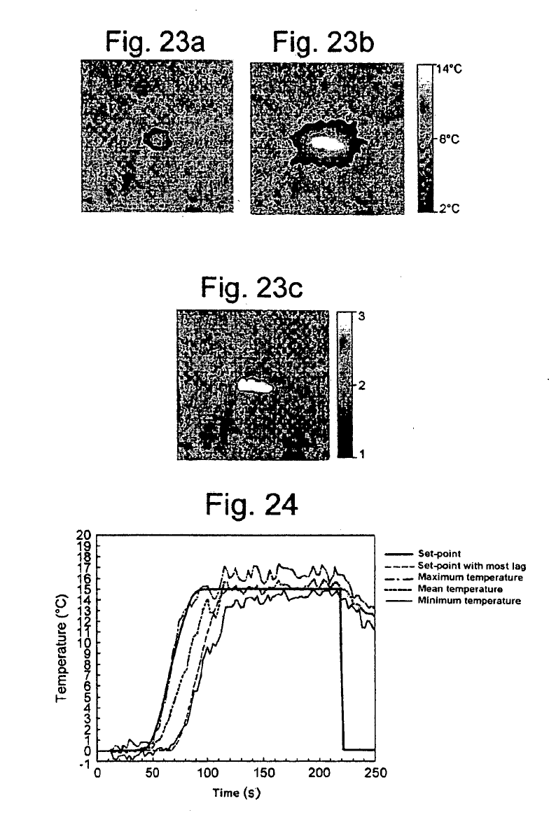

FIGS. 23a and 23b show the two thermal maps obtained in the middle and at the end of the temperature regimen i.e. at dynamics 32 and 48 respectively. As expected, the central point was heated first, then the adjacent points to achieve final uniform heating of a desired 11 mm segment. As always, heating was extended through thermal diffusion, but this time to a lesser extent along axis X at the end of the segment, compared with the thermal maps shown FIGS. 19a and 19b. The necrosis obtained at the end of heating 11×4 mm², shown FIG. 23c, is similar to that previously obtained with the only difference that it is slightly shorter by one millimetre.

The statistical study (see FIG. 24) of the minimum, mean and maximum temperature of the 11 controlled voxels is slightly different since the temperature regimen was not the same for all points. Therefore during the temperature rise, the central point systematically corresponds to the hottest point, and the two peripheral points to the coldest points. During this phase, the difference between the maximum and minimum temperature is widened so as to correspond to the difference between the central regimen and the one the most offset. Once the temperature rise is completed, this difference with the maximum temperature returns to 2° C. as previously. Also the temperature hold of 15° C. is followed to a mean accuracy of 0.5° C.

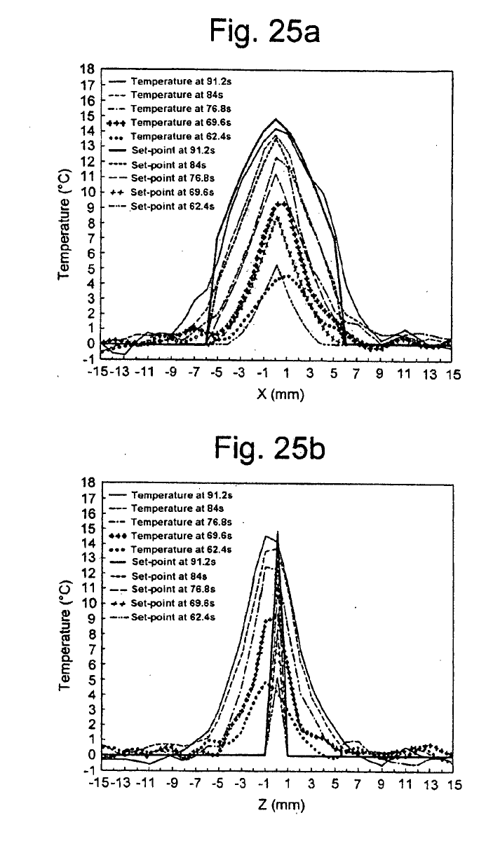

To observe the flexibility with which the spatial control algorithm can control temperature, 5 of the triangular regimens and corresponding temperatures obtained are shown FIGS. 25a and 25b. The spatial distribution of temperature precisely follows the set temperature irrespective of their shape, for as long as the imposed temperature gradients do not exceed those produced by thermal diffusion. In this example with a maximum slope of 2° C./mm, this limitation is not a hindrance. Additionally the temperature outside the volume, heated along the X axis, is lower since it does not exceed 4° C. at time 90 s. The widening of the heated volume along the Z axis on the other hand is slightly greater than previously, since the temperature rise at the central point occurs more rapidly.

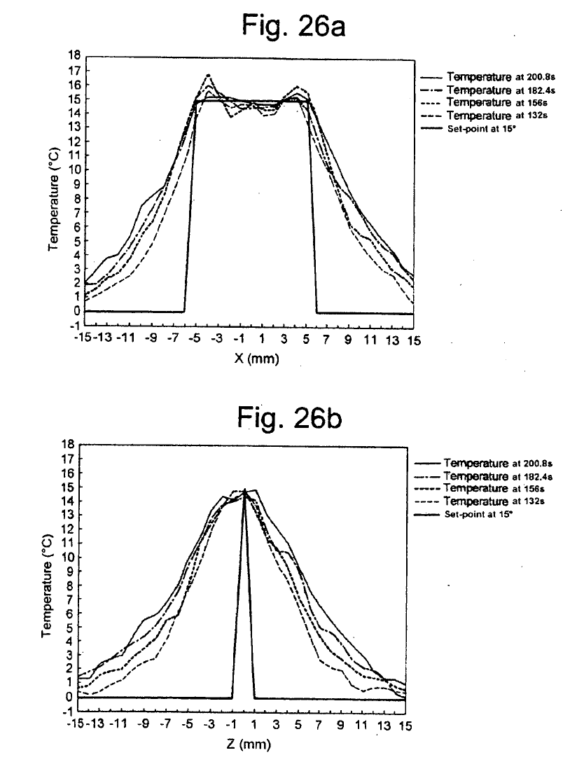

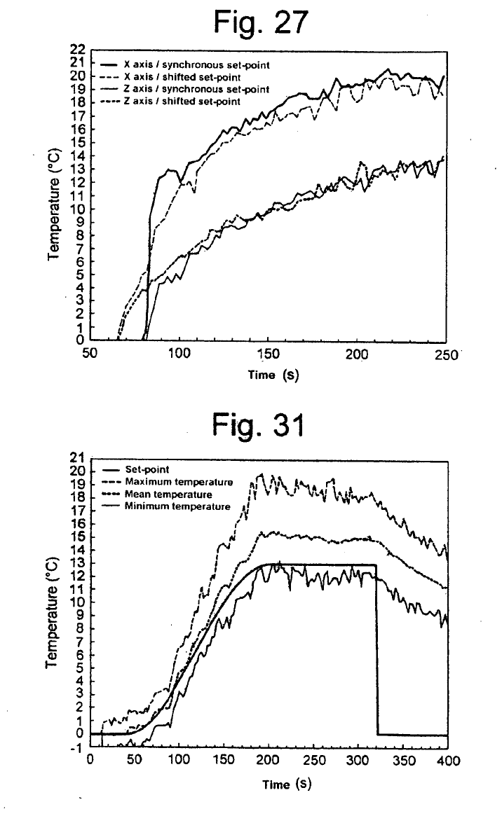

As shown FIGS. 26a and 26b, the temperature is maintained constant and homogeneous over the heated volume, only the outside temperature increases gradually. To evaluate this extension of heating through thermal diffusion and to compare the two preceding experiments, FIG. 27 gives the width at mid-height i.e. at 7.5° C., of the heated volume along axes X and Z.

During the temperature rise from 60 s to 120 s, the widths at mid-height in the two experiments are very different. The heating applied with synchronized temperature setting causes all the points simultaneously to overstep the value of 7.5° C., which explains the sudden increase in width at mid-height at 80 s. On the other hand, heating applied with shifted temperature setting first brings the central temperature to above 7.5° C., and then the adjacent points from one to another, which induces a progressive increase in the width at mid-height up to 11 mm. It is then thermal diffusion which increases the width at mid-height. As a result, the width at mid-height along axis X when heating with shifted temperature setting is 1 mm smaller than that obtained with synchronized setting, since the off-centre points are heated later. Along the Z axis, the opposite phenomenon is observed to a lesser extent, since the central point is heated more quickly. As heating progresses, these differences diminish. This accounts for the size of the necrosis obtained using shifted setting, this necrosis being one millimetre longer and of identical width to that obtained with synchronized setting.

3D Cubic Temperature Control

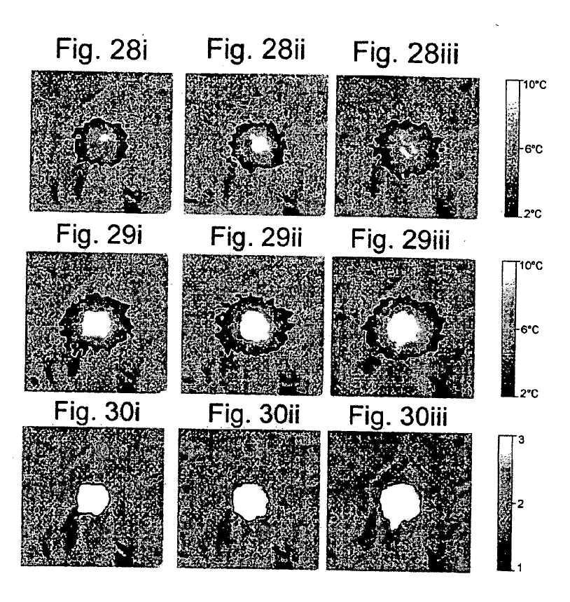

To evaluate the performance of the temperature control algorithm on a 3D volume, the cube of 147 points previously described in FIGS. 13i, 13ii and 13iii, was heated with a synchronized regimen of 12° C. for 220 s to obtain a thermal dose over the entire volume. The results obtained for this experiment are given FIGS. 28, 29 and 30.

The temperature maps for the 3 slices of interest indicate cubic heating of 6° C. then 10° C. as expected. The square shape is distinctly apparent in slice 3 (lying deepest in the tissue) and is seen to become more and more rounded in slices 4 and 5. The same applies to the necrosis obtained at the end of heating, which is in the shape of a square with sides of 8 mm, and becomes progressively rounder in slices 4 and 5.

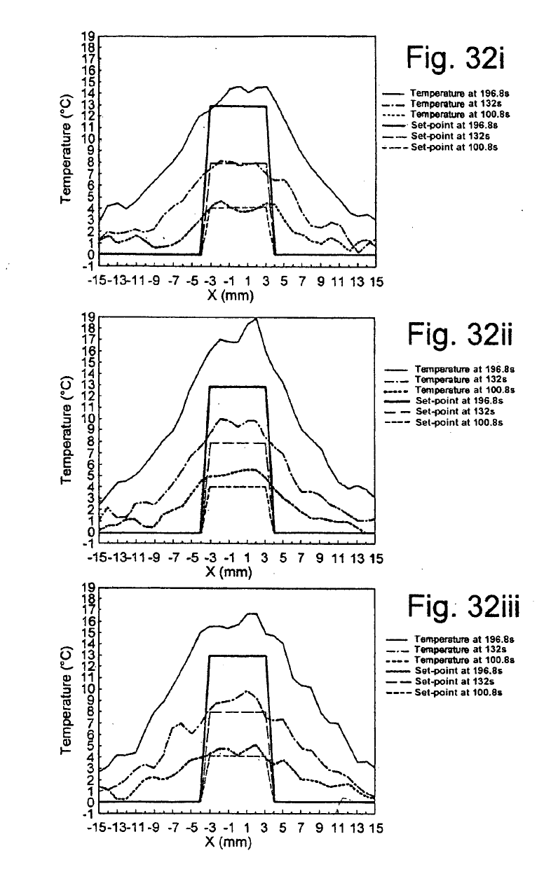

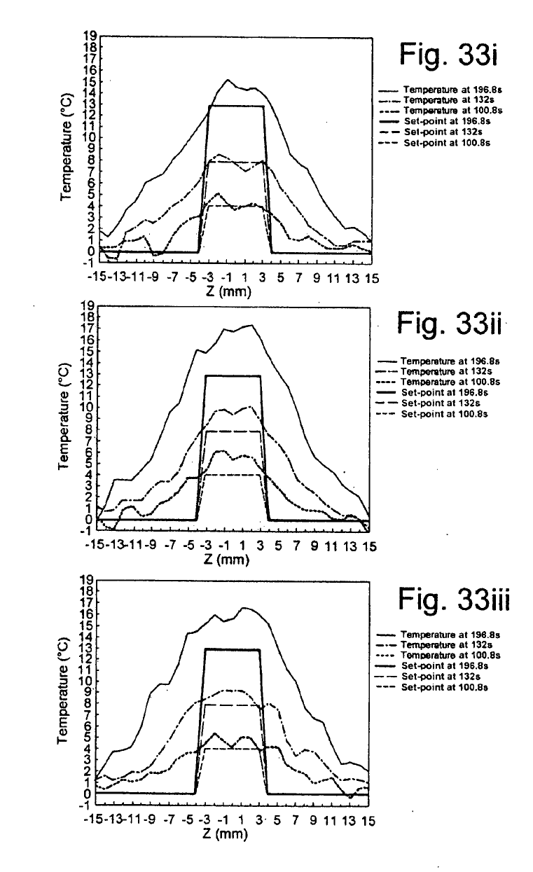

Statistical analysis of the minimum, mean and maximum temperature over the entire heated volume, shown FIG. 31, indicates greater differences than those previously obtained. The difference between the maximum temperature and the minimum temperature is effectively an average of 6° C. instead of 2° C. since there are 147/11=13 times more controlled points. The influence of noise on this difference is therefore more than 3 times greater. On the other hand, the calculated mean temperature is all the less affected by noise. However, it clearly indicates a constant overstepping by 3° C. of the set temperature. This effect derives from the PID control algorithm and the algorithm for detection of maximum required energy, which force the coldest voxel to reach the set temperature at the expense of the voxels that are too hot. In addition, the minimum temperature voxel systematically lies on slice 3 and the maximum temperature voxel on slice 4. To observe this effect in more detail, FIGS. 32 and 33 show the heating achieved on the three central slices along axes X and Z respectively, at the start, in the middle and at the end of the temperature rise.

The set temperature is precisely attained on slice 3. However, this slice is globally colder than slices 4 and 5. The temperature measured on these two other slices effectively exceeds the set temperature. On slice 4, the hottest slice, this overstepping can reach 6° C.

Through the superimposition effect of the different ultrasound propagation cones, the heating of a surface induces heating 1.3 times longer in length. Therefore each heating applied to slice 3 induces equivalent heating on slices 4 and 5. With the additional attenuation effect of the ultrasound wave when passing through the tissue, the energy arriving on slice 3 (the one lying deepest in the tissue) is lower than the energy which passed through slices 4 and 5. As a result, only the temperature on slice 3 is controlled, since heating on the deepest lying slice induces greater heating on the other slices. As detailed in Table 1, 85% of the total energy emitted is focused on slice 3.

TABLE 1
Depth distribution of deposited energy with cubic heating

| | Slice 3 | Slice 4 | Slice 5 |
|---|---|---|---|
| Deposited energy | 85.25% | 8.03% | 6.72% |

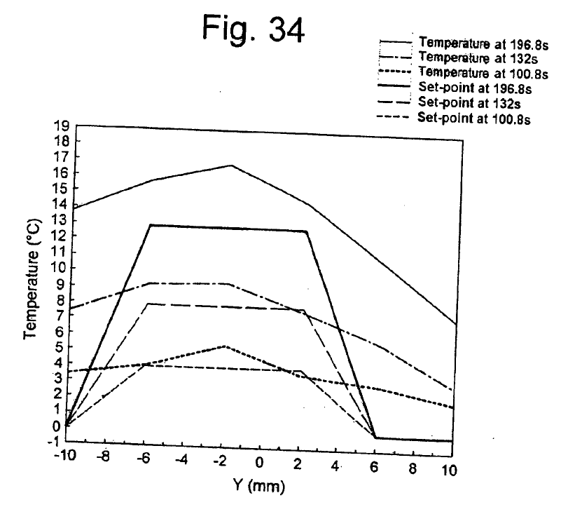

Despite all this, as shown FIG. 34 illustrating the temperature measured along axis Y, the maximum lies in slice 4. Therefore, outside slices 3, 4 and 5 the temperature decreases. Evidently this decrease remains very limited, especially in slice 6 between the transducer and the heated volume. The necrosis obtained in this slice is of equivalent size to that obtained in the other slices. Tissue absorption and the overlap effect impose the obtaining of rather elongate heating towards the energy source, the transducer. In practice, this consequence is harmful for treatment safety, since it can cause skin burns if treatment is applied less than 15 mm away from the skin.

Despite these physiological and material constraints, PID control allows definition of the energy that is strictly necessary to necrotize the deepest lying slice. The algorithm can be further optimized using a model shape of the focusing point which takes into account the coefficient of absorption and tissue attenuation, but even without this the desired result is obtained.

TABLE 2
Geometric distribution of deposited energy with cubic heating

| | Corners | Edges | Other |
|---|---|---|---|
| Deposited energy | 63% | 33.3% | 3.7% |

The study on geometric distribution of deposited energy (Table 2) indicates that two thirds of total energy are located in the corners, one third on the edges and a negligible percentage in the centre. This distribution is similar to the point density obtained with the detection of maximum algorithm in FIGS. 16a to 16c. Generally, for a 3D topology, the focusing required inside the volume is very low since it is indirectly heated through thermal diffusion from peripheral focusing points. On this account, the use of a 3D-shifted temperature regimen gives no notable difference on the heating produced.

Spherical 3D Temperature Control

The obtaining of cubic heating is theoretically most instructive, but does not truly correspond to the shape of tumours which are generally spherical. Additionally, almost all the energy is deposited in the corners and edges of the cube. To obtain a more homogeneous energy deposition, and more realistic heating, the preceding experiment was reproduced using spherical heating. The temperature time setting was identical, but the corners and edges of the cubes were removed from the spatial setting.

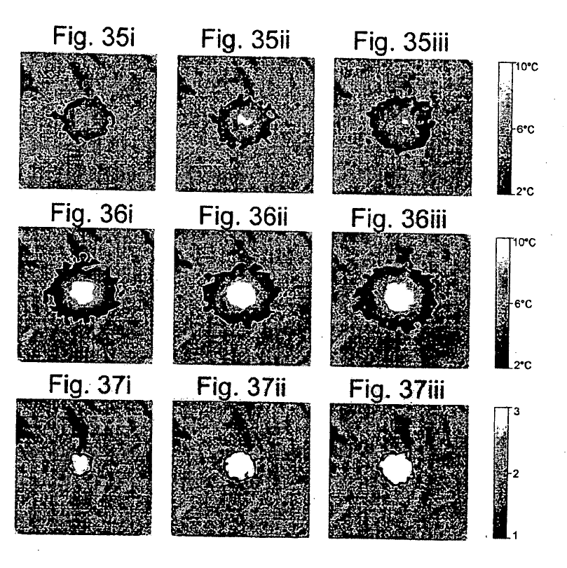

As with the other experiments, two temperature maps obtained in the middle and at the end of temperature rise, and the dose produced at the end of heating are given in FIGS. 35 and 36. Given the newly chosen spherical shape, the temperature maps and thermal dose obtained are rounded with greater or lesser diameters depending on the observed slice. Only the diameter of heating and of the necrosis produced on slice 5 (FIG. 37iii) is slightly greater than planned. Also, heating was fairly homogeneous on the target volume except in slice 4 which was a little hotter in the centre of the sphere. As with each heated volume, the central temperature is higher taking into account the effect of thermal diffusion and the superimposition of the acoustic beams.

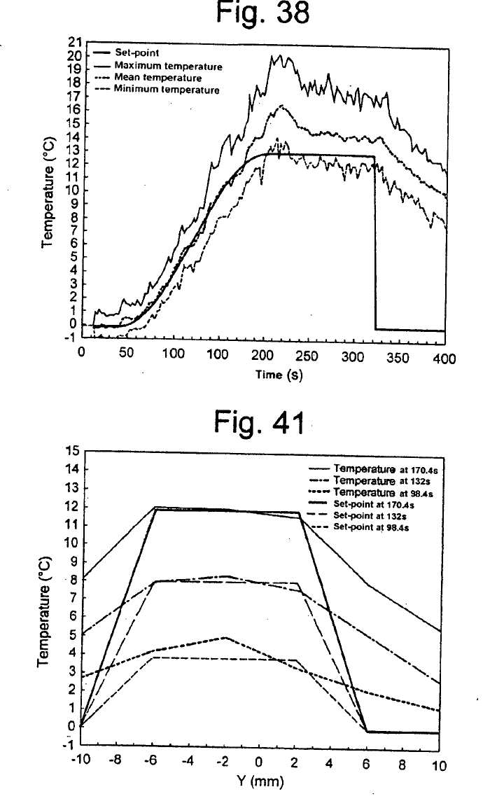

The difference between the maximum and minimum temperature on the controlled volume shown FIG. 38 is now 5° C. This difference, although smaller than previously, still remains fairly high since firstly the number of controlled voxels, 89, is much higher than the number of points covered per trajectory, and secondly the desired spherical shape does not belong to strictly feasible shapes. The volumes which are feasible with this transducer geometry are rather more an elongate ellipsoid whose height to width ratio must be equal to or greater than 1.3. However, the spherical shape is nonetheless more feasible than the cubic shape, and consequently the overstepping of the set temperature by the mean temperature is 2° C. instead of 3° C. as previously.

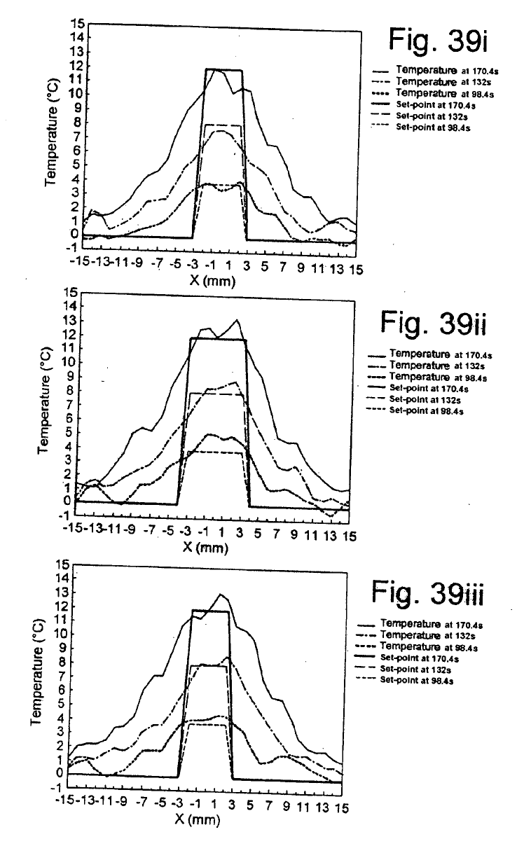

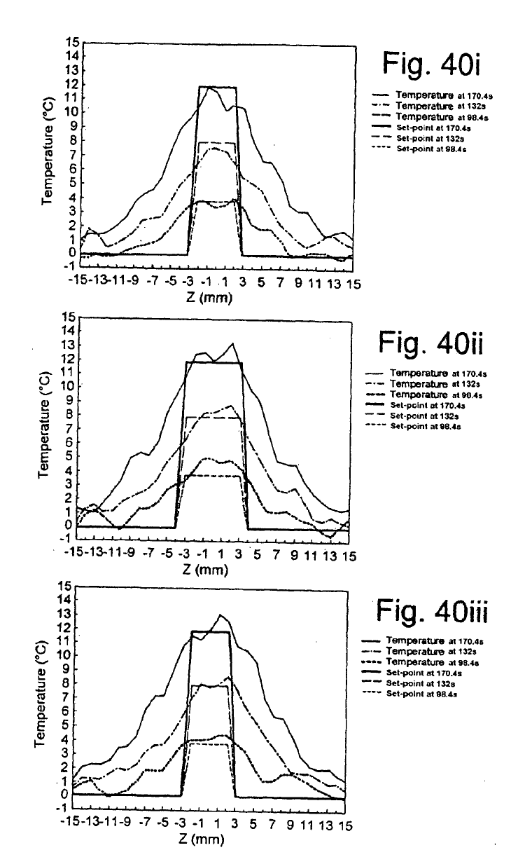

FIGS. 39 and 40 show the temperatures measured along axes X and Z in more detail, during the temperature rise. As previously, slice 3 is the coldest and slice 4 the hottest. On the other hand, the heating achieved is much closer to the set temperature. Although the tissue and the transducer are identical, the attenuation effect on passing through the tissue and the superimposition effect of the propagation cones are less extensive. Attenuation is lesser since the deepest lying slice 3 comprises 21 points instead of 47 previously. The overlap is less extensive since the points the furthest away from the centre, the corners and edges of the cube were removed from the spatial setting. The depth distribution of deposited energy is now more homogeneous.

TABLE 3
Depth distribution of deposited energy with spherical heating

| | Slice 3 | Slice 4 | Slice 5 |
|---|---|---|---|
| Deposited energy | 46.88% | 49.5% | 3.62% |

As detailed Table 3, the focusing points are distributed more or less equally between slice 3 and slice 4. This choice leads to the desired heating of slice 5, even though it contains practically no focusing point. On the other hand, since this slice was heated by focusing on the upper slices, the area which is heated cannot have a smaller diameter than in slice 4. On this account, the final necrosis obtained in slice 5 is much greater than defined in the spatial temperature regimen.

FIG. 41 indicates the central temperature measured along axis Y and confirms the good homogeneity of heating between slices 3, 4 and 5. In addition, the temperature decreases more rapidly on the other slices since the heated volume is of smaller size. Also, it is not by chance that the temperature in slice 3 is slightly below the set temperature at the start of the temperature rise, as previously in FIG. 31. This error related to the non-fulfillment of the shape of the focusing point with respect to the coefficient of attenuation is rapidly compensated by the PID control algorithm.

To conclude, when the overlapping of the different focal points is less extensive, spatial temperature control can be made by likening required energy to the point density to be applied. This is notably the case with multispiral 2D spatial control, since all the focal points lie in the coronal plane which is perpendicular to the axis of propagation of the wave.

On the other hand, when the imaging plane includes the axis of propagation of the ultrasound wave, the different focusing points overlap.

Approximation of required energy by point density is then no longer valid since it produces energy that is too high. This overstepping of the energy produced is most marked during 3D temperature control, and it is therefore essential to take into account the shape of the focusing point to quantify the overlap effect.

By associating the detection of maximum algorithm with PID spatial control to take the shape of the focal point into account, 3D temperature control then becomes possible. The temperature can subsequently be controlled over the entire treated volume.

The described 3D retroaction system is intended to control the minimum temperature rather than the mean temperature of the set volume. This is practical for ablation of a malignant tumour since necrosis is therefore guaranteed on each voxel of the volume. On the other hand, for non-destructive use such as for activation of a heat-sensitive liposome, this control should be modified to ensure, instead, a maximum temperature that is not to be exceeded. Control which achieves both simultaneously would evidently be ideal. This is more or less feasible depending on the size and shape of the heated volume. A segment of a few points is a fairly close example. To obtain precise control over an entire volume irrespective of its shape, ideally a transducer should be provided with a large aperture angle to reduce the overlap effect. This technological limitation, although it can be improved upon, will never be perfect. It is therefore advisable, before starting treatment, to conduct a simulation of induced heating to anticipate and adjust the volume of ablation.

The reader will appreciate that numerous modifications may be made hereto without departing materially from the new teachings and advantages described herein. Therefore, all modifications of this type are intended to be incorporated within the scope of the thermal treatment device for biological tissue, as in the invention, and the scope of the associated thermal treatment method.

1. A thermal treatment device to treat a target region of a biological tissue, comprising:
energy generating means (3,4) to supply energy to a focal point (P) in the target region,
means (2) to measure the spatial distribution of temperature in said region,
a control unit (7) comprising means to control moving of the focal point (P) towards successive treatment points, and means to control the energy to be provided by the generator means (3,4) to the successive treatment points,
wherein the control unit (7) further comprises means to determine a spatial and energy distribution of the treatment points to be performed, each successive treatment point having a determined position and treatment energy in relation to the positions and spatial energy distributions of the previous treatment points.

2. The device of claim 1, wherein the spatial distribution of the treatment points is three-dimensional.

3. The device of claim 1, wherein the control unit comprises means to determine the spatial and energy distribution of the treatment points to be performed in relation to a spatial distribution of required energy defined by a temperature regulation system to treat the target region.

4. The device of claim 3, wherein the control unit comprises means to determine the spatial distribution of required energy to treat the target region according to a Proportional-Integral-Derivative regulation system.

5. The device of claim 3, wherein the means to determine the spatial and energy distribution of the treatment points to be performed comprise deconvolution means of the spatial distribution of required energy to treat the target region, using a spatial energy distribution characteristic of one treatment point.

6. The device of any of claim 3, wherein the means to determine the spatial and energy distribution of the treatment points to be performed comprise means to determine the treatment point corresponding to the maximum spatial distribution of remaining energy to treat the target region, the spatial distribution of remaining energy corresponding to the spatial distribution of energy required to treat the target region subtracted by the spatial energy distributions characteristic of the previous treatment points.

7. The device of claim 6, wherein the control unit comprises means to determine the spatial and energy distribution of the treatment points to be performed, such that:
$${E}^{0} ( \vec{r} ) = {E}^{REQUIRED} ( \vec{r} ) and {Γ}^{0} ( \vec{r} ) = 0$$
$$\vert \begin{matrix}let {\vec{r}}_{n} such that {E}^{n} ( {\vec{r}}_{n} ) = max ( {E}^{n} ( \vec{r} ) ) \\ {E}^{n + 1} ( \vec{r} ) = {E}^{n} ( \vec{r} ) - R \cdot {E}^{n} ( {\vec{r}}_{n} ) \cdot {E}^{1 pt} ( \vec{r} - {\vec{r}}_{n} ) \\ {Γ}^{n + 1} ( \vec{r} ) = {Γ}^{n} ( \vec{r} ) + R \cdot {E}^{n} ( {\vec{r}}_{n} ) \cdot δ ( \vec{r} - {\vec{r}}_{n} )\end{matrix}$$
in which Eⁱ(\vec{r}) corresponds to the difference between the spatial distribution of required energy and the spatial energy distributions characteristic of the previous i treatment points \vec{r}ᵢ, Γⁱ(\vec{r}) corresponds to the spatial distribution of the associated treatment points, δ is the Dirac function which is zero at every point \vec{r} different from the origin where its value is 1, R is a percentage chosen arbitrarily so that the spatial energy distribution characteristic of a treatment point is less than the spatial distribution of required energy E^{REQUIRED}(\vec{r}).

8. The device of claim 1, wherein the control unit comprises means to control the energy supplied by the energy generating means in relation to a non-uniform temperature set point.

9. The device of claim 1, wherein the control unit comprises means to control the energy supplied by the energy generating means in relation to a time-shifted temperature set point for each treatment point.

10. A thermal treatment method to treat a target region of a biological tissue, comprising the steps of:
measuring the spatial temperature distribution in said region,
controlling movement of a focal point (P) towards successive treatment points in the target region,
providing energy to the focal point (P),
characterized in that it further comprises a step of determining a spatial and energy distribution of the treatment points to be performed, each successive treatment point having a position and a treatment energy determined in relation to the positions and spatial energy distributions of the previous treatment points.
